(19) United States
(12) Patent Application Publication
(10) Pub. No.: US 2002/0068711 A1
(43) Pub. Date: Jun. 6, 2002

(54) Arrest of proliferation of highly glycolytic tumors

(76) Inventors: Peter L. Pedersen, Columbia, MD (US); Saroj P. Mathupala, Clinton Twp, MI (US)

Correspondence Address: Lisa A. Haile, J.D., Ph.D., GRAY CARY WARE & FREIDENRICH LLP, 4365 Executive Drive, Suite 1600, San Diego, CA, 92121-2189, US

(21) Appl. No.: 09/808,743

(22) Filed: Mar. 14, 2001

Related U.S. Application Data
(60) Provisional application No. 60/189,222, filed on Mar. 14, 2000.

(51) Int. Cl. A61K 48/00

(52) U.S. Cl. 514/44

(57) ABSTRACT

The present invention relates to a method of inhibiting the proliferation of tumor cells using antisense polynucleotides or oligonucleotides. Tumor cells having a highly glycolytic phenotype can be inhibited with an antisense molecule that hybridizes with a nucleic acid encoding a hexokinase. The present invention also relates to recombinant nucleic acid molecules useful for regulating transcription and translation and which can contain an antisense molecule. The present invention also relates to pharmaceutical preparations containing antisense molecules.

### RELATED APPLICATIONS

This application claims the benefit of priority under 35 U.S.C. § 119(e) to Provisional Application Serial No. 60/189,222, filed Mar. 14, 2000, the content of which is incorporated by reference in its entirety herein.

### GOVERNMENT SUPPORT

The invention described herein were made in part with government support under Grant CA 80118 awarded by the National Institutes of Health. The government may have certain rights in this invention.

### FIELD OF THE INVENTION

The present invention relates to methods of inhibiting the proliferation of tumor cells having a highly glycolytic phenotype using antisense polynucleotides or oligonucleotides that hybridize to nucleic acids encoding hexokinase.

### BACKGROUND OF THE INVENTION

One of the most common, profound, and intriguing phenotypes of highly malignant tumors, known for more than six decades, is their ability to metabolize glucose at high rates. This is a characteristic of both animal and human tumors including those derived from brain, breast, colon, liver, lung, and stomach. In each, a close correlation exists among the degree of de-differentiation, growth rate, and glucose metabolism, where the most de-differentiated tumors exhibit the fastest growth and the highest glycolytic rate. In fact, this unique phenotype is used clinically worldwide in Positron Emission Tomography (PET) to detect tumors, assess their degree of malignancy, and in some, cases even predict survival time.

Despite the commonality of the high glycolytic phenotype, and its widespread use clinically as a diagnostic tool, it has not been exploited as a major target for arresting or slowing the growth of cancer cells. This is because until very recently the underlying molecular basis of the high glycolytic phenotype remained unknown. It had long been suspected to involve some type of glycolytic mitochondrial interaction but the mechanism was not known. Recent experiments have directly demonstrated a requirement for an overexpressed mitochondrially bound form of hexokinase, now identified as Type II hexokinase.

Antisense technology is emerging as an effective means of lowering the levels of a specific gene product. It is based on the findings that these “antisense” sequences hybridize a gene or associated target polynucleotide, to form a stable duplex or triplex, based upon Watson-Crick or Hoogsteen binding, respectively. The specifically bound antisense compound then either renders the respective targets more susceptible to enzymatic degradation, blocks translation or processing, or otherwise blocks or inhibits the function of a target polynucleotide. Where the target polynucleotide is RNA, the antisense molecule hybridizes to specific RNA transcripts disrupting normal RNA processing, stability, and translation, thereby preventing the expression of a targeted gene. Administration of antisense polynucleotides complementary to the mRNA of interest have been shown to block the translation of specific genes in vitro and in vivo. Antisense technology may therefore be useful in inhibiting highly glycolytic tumor by blocking translation of key genes required for proliferation of tumor cells.

### BRIEF DESCRIPTION OF THE DRAWINGS

FIG. 1 shows the construction of the pLXRN bicistronic retroviral vector backbone. The positions of the restriction enzyme sites (BamHI and RsrII) are indicated. LTR, long terminal repeats (viral promoter regions); Neomycin, neomycin phosphotransferase cDNA, MCS (multiple cloning site).

FIG. 2 shows the primary sequence of the type II hexokinase from AS-30D hepatoma cells. The amino acid positions are indicated to the left of figure. Arrow indicates the direction of translation and the coding region.

FIG. 3A shows a SDS-PAGE profile for the whole cell extracts from sham-transfected and recombinant retroviral vector transfected AS-30D hepatoma cell clones. (10 μg of total protein per lane). Lane 1, Biotinylated molecular weight markers; Lane 2, Sham-transfected cells; Lane 3, pLXRN retrovirus transfected Control cells; Lane 4, Recombinant pLXRN-Anti-sense Type II Hexokinase cDNA retrovirus transfected Antisense clonal isolate showing a 93% reduction in hexokinase activity; Lane 5, Bio-Rad High Molecular Weight markers. The positions of molecular weight markers (200, 116, and 97 kDa) are indicated.

FIG. 3B shows a Western Blot profile of the whole cell extracts from sham-transfected and recombinant retroviral vector transfected AS-30D hepatoma cell clones. A duplicate blot of the SDS-PAGE profile shown in A (10 μg total cell extract per lane) was probed using a polyclonal antibody generated against Type II Hexokinase. Lane 1, Biotinylated molecular weight markers; Lane 2, Sham-transfected cells; Lane 3, pLXRN retrovirus transfected Control cells; Lane 4, Recombinant pLXRN-Anti-sense Type II Hexokinase cDNA retrovirus transfected Antisense clonal isolate showing a 93% reduction in hexokinase activity; Lane 5, Bio-Rad High Molecular Weight markers. The positions of molecular weight markers (200, 116, and 97 kDa) are indicated.

FIG. 4 shows the proliferative capacity of sham-transfected, control, and hexokinase anti-sense clonal isolates. Total CPM from ³H-thymidine incorporated per unit total protein are shown: Row 1, Anti-sense cells; Row 2, Control cells; Row 3, Sham-transfected cells.

FIG. 5 shows residual hexokinase activity of the anti-sense hexokinase clonal isolate in contrast to control and sham-transfected cells. The hexokinase activities of sham-transfected (118%) and anti-sense cells (7%) are shown against the activity in control cells adjusted to 100%.

FIG. 6 shows the complete nucleotide sequence of Rattus norvegicus mutant type II hexokinase mRNA, Accession Number AF027179 (SEQ ID NO:1).

FIG. 7 (A, B and C) shows the complete nucleotide sequence of cloning vector pLXRN, Accession number AF113968 (SEQ ID NO:3).

FIG. 8 (A and B) shows the nucleic acid sequence of Rattus norvegicus type I hexokinase (HKI) mRNA, Accession number NM 012734 (SEQ ID NO:2).

### SUMMARY OF THE INVENTION

The invention provides a method of inhibiting proliferation of tumor cells characterized by having a highly glycolytic phenotype. The method includes contacting tumor cells with an effective amount of an antisense oligonucleotide or polynucleotide that hybridizes with a nucleic acid encoding a hexokinase.

The invention also provides pharmaceutical preparations that are useful for inhibiting proliferation of tumor cells. The preparations contain antisense oligonucleotides and polynucleotides that hybridizes with nucleic acid encoding a hexokinase.

The invention also provides recombinant nucleic acid vectors containing multiple elements operably linked in a 5′ to 3′ direction. The elements include a 5′ and a 3′ long terminal repeat (LTR), a multiple cloning site (for restriction enzymes EcoRI, HpaI, XhoI and BamHI), an internal ribosome entry sequence (IRES) and a selectable marker sequence.

The invention further provides a recombinant nucleic acid molecule containing a polynucleotide complementary to a nucleotide sequence encoding Type II hexokinase.

### DETAILED DESCRIPTION OF THE INVENTION

The present invention provides methods of inhibiting proliferation of highly glycolytic tumor cells using antisense molecules, pharmaceutical preparations useful to such inhibition, and recombinant nucleic acid vectors containing antisense molecules. The antisense molecule is able to hybridize to messenger ribonucleic acids (mRNA) encoding hexokinase, an enzyme that plays a key role in initiating the glycolytic cascade.

The present invention employs oligomeric antisense compounds, particularly oligonucleotides, for use in modulating the function of nucleic acid molecules encoding a hexokinase, ultimately modulating the amount of hexokinase produced. This is accomplished by providing antisense compounds which specifically hybridize with one or more nucleic acids encoding hexokinase. As used herein, the terms “target nucleic acid” and “nucleic acid encoding a hexokinase” encompass DNA encoding a hexokinase, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of a hexokinase. In the context of the present invention, “modulation” means either an increase (stimulation) or a decrease (inhibition) in the expression of a gene. In the context of the present invention, inhibition is the preferred form of modulation of gene expression and mRNA is a preferred target.

It may be preferred to target specific nucleic acids for antisense. “Targeting” an antisense compound to a particular nucleic acid, in the context of this invention, is a multi-step process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a hexokinase. The targeting process can also include determination of a site or sites within the nucleic acid molecule for the antisense interaction to occur such that the desired effect, e.g., detection or modulation of expression of the protein, will result. In the present invention, preferred sites are the regions important for transcription initiation, translation initiation, and the proximal promoter region. The translation initiation codon is typically 5′-AUG (in transcribed mRNA molecules; 5′- ATG in the corresponding DNA molecule), the translation initiation codon is also referred to as the “AUG codon,” the “start codon” or the “AUG start codon.” A minority of genes have a translation initiation codon having the RNA sequence 5′- GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokayotic genes may have two or more alternative start codons, any one of which may be preferentially utilized for translation initiation in a particular cell type or tissue, or under a particular set of conditions. In the present invention, “start codon” and “translation initiation codon” refer to the codon or codons that are used in vivo to initiate translation of an mRNA molecule transcribed from a gene encoding a hexokinase, regardless of the sequence(s) of such codons. In one embodiment of the present invention, a sequence complementary to the entire cDNA is used. Such an antisense molecule can target either the mature mRNA, or the individual exons within the pre-mRNA.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a pre-mRNA transcript to yield one or more mature mRNA. The remaining (and therefore translated) regions are known as “exons” and are spliced together to form a continuous mRNA sequence. mRNA splice sites, i.e., exon-exon or intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. Targeting particular exons in alternatively spliced mRNAs may also be preferred. It has also been found that introns can also be effective, and therefore preferred, target regions for antisense compounds targeted, for example, to DNA or pre-mRNA.

As used herein “tumor cells characterized by having a highly glycolytic phenotype” and “highly glycolytic tumor cells” refer to tumors that display a high rate of glycolysis. Tumor cells characterized by having a highly glycolytic phenotype metabolize glucose at high rates compared to normal cells. The elevated rate of glucose catabolism is important for highly malignant tumors which obtain over 50% of their energy and the anabolic precursors for biosynthetic pathways, via glycolysis. “Tumor cells” refers to cells that are local or a part of the solid tumor or cells that are metastatic. Thus, the method of the invention is contemplated for treating or inhibiting the growth of cells that are part of a primary tumor and for treating or inhibiting the growth of cells that are metastatic. It should be understood that cells having metastatic potential within a primary tumor are included in the description.

Any tumor cell having a highly glycolytic phenotype is susceptible to treatment by invention methods. Tumor cells can be located in a tissue selected from brain, colon, urogenital, lung, renal, prostate, pancreas, liver, esophagus, stomach, hematopoietic, breast, thymus, testis, ovarian, skin, bone marrow and uterine tissue.

Any cellular proliferative disorder in which proliferative cells have a highly glycolytic phenotype are susceptible to treatment by invention methods. Exemplary cellular proliferative disorders include low grade astrocytoma, anaplastic astrocytoma, glioblastoma, medulloblastoma, gastric cancer, hepatoma, colorectal cancer, colorectal adenoma, acute myelogenous leukemia, lung cancer, renal cancer, leukemia, breast cancer, prostate cancer, endometrial cancer, bone cancer, squamous cell cancer and neuroblastoma. Although cells in leukemia and other hematopoietic cell proliferative disorders may not be considered “tumor cells” per se, such cells are included in the methods of the invention and the description of “tumor cells” as used herein.

Prior to treating tumor cells with the antisense polynucleotide or oligonucleotide, a cellular proliferative disorder can be diagnosed by methods known in the art. For example, the cells are diagnosed as highly glycolytic by obtaining a specimen of serum, urine, saliva, blood, cerebrospinal fluid, pleural fluid, ascites fluid, sputum, stool, bone marrow or biopsy sample.

As used herein, “treating” refers to inhibiting or arresting the development of a disease, disorder or condition and/or causing the reduction, remission, or regression of a disease, disorder or condition. Those of skill in the art will understand that various methodologies and assays may be used to assess the development of a disease, disorder or condition, and similarly, various methodologies and assays may be used to assess the reduction, remission or regression of a disease, disorder or condition.

As employed herein, the phrase “a therapeutically effective amount”, when used in reference to invention methods employing antisense compounds and pharmaceutically acceptable salts thereof, refers to a dose of compound sufficient to provide concentrations high enough to impart a beneficial effect on the recipient thereof. As used herein, “an inhibitory effective amount” refers to a dose of invention compounds sufficient to provide concentrations high enough to inhibit proliferation of tumor cells having a highly glycolytic phenotype. The specific therapeutically effective dose level for any particular patient will depend upon a variety of factors including the disorder being treated, the severity of the disorder, the activity of the specific compound used, the route of administration, the rate of clearance of the specific compound, the duration of treatment, the drugs used in combination or coincident with the specific compound, the age, body weight, sex, diet and general health of the patient, and like factors well known in the medical arts and sciences. Dosage levels typically fall in the range of about 0.001 up to 100 mg/kg/day; with levels in the range of about 0.05 up to 10 mg/kg/day being preferred.

In comparison to normal cells, expression of the enzyme, hexokinase, is markedly elevated in highly glycolytic, rapidly growing tumors (Pedersen (1978) Prog. Exp. Tumor Res., 22:190-274; and Mathupala et aL, (1997) J. Bioenergenics and Biomembranes 29:339-343, each of which is incorporated by reference herein). As used herein, “hexokinase” refers to a family of enzymes, each of which commits glucose to catabolism in the first step of the glycolytic pathway. Certain isozymes of hexokinase are over-expressed in rapidly growing, highly glycolytic tumors.

Hexokinase (ATP: D-Hexose-6-phosphotransferase) catalyzes the following reaction:

Glucose+ATP⇄Glucose-6-PO₄,+ADP

There are four major isozymes of hexokinase, referred to as Type I, II, III and IV (glucokinase). Types I to III show a high affinity (Kₘ,=0.02-0.03 mM) for glucose, are product inhibited by glucose-6-PO₄ and have a molecular mass near 100 kDa. In contrast, the Type IV isozyme (glucokinase) has a 100-fold lower affinity for glucose (Kₘ,=5−8 mM), is insensitive to product inhibition by glucose-6-PO_{4,} and has a 20 molecular mass near 50 kDa (See Table 1).

TABLE 1

| | | | | Production |
|---|---|---|---|---|
| | | | Molecular | Inhibition |
| Hexokinase | Km | Major Tissue | Mass | (by |
| Type | (mM) | Location | (kDa) | Glucose-6-PO₄) |
| I | Low | Brain, Kidney | 100 | + |
| II | Low | Muscle, | 100 | + |
| | | Adipose | | |
| III | Low | Low in Several | 100 | + |
| | | Tissues | | |
| IV | High | Liver, Pancreas | 50 | − |

Within the last ten years cDNAs encoding all four hexokinase isozymes have been cloned and sequenced from their major tissues of origin (See, Thelan et al. (1991) Arch. Biochem. Biophys., 286: 645-651 (for Type II), Nishi et al., (1988) Biochem. Biophys. Re. Commun. 157:937-943, Schwab and Wilson (1989) Proc. Natl. Acad. Sci. USA 86:2563-2567; Scwab and Wilson (1991) Arch Biochem Biophys. 285:365-370; and Andreone et al. J. Biol. Chem. 264:363-369). Hexokinase isozymes from three different tumors exhibiting the high glycolytic phenotype have been cloned and sequenced also (Thelan and Wilson (1991) Arch. Biochem. Biophys. 286:645-651; Arora et al. (1990) J. Biol. Chem. 265:6481-6488; and Mathupala and Pedersen (1997) Gen Bank Accession Number AF027179, each of which is herein incorporated by reference in its entirety). One of the deduced sequences confirms that the major hexokinase isozyme expressed in such tumors is Type II, which is found normally in muscle and adipose tissue in low amounts. Type I, found normally in brain and kidney, is found also in some highly glycolytic tumors, but at very low levels relative to the markedly overexpressed Type II isozyme. Possible exceptions are certain brain tumors (Rijksen and Staal (1985) Hexokinase in Health and Disease. In: Regulation of Carbohydrate Metabolism (Breitner, R., ed., CRC Press I, 89-99).

Type II hexokinase binds to transmembrane channels formed by the protein called “porin” or “VDAC” located within the outer mitochondrial membrane. This interaction markedly reduces the enzyme's sensitivity to product inhibition by glucose6-PO₄, provides preferred access to mitochondrially generated ATP, and protects the hexokinase against proteolytic degradation. These combined properties, together with the high content of the enzyme in highly malignant tumors (>100 fold elevation), result in the rapid production of glucose-6-PO₄. This key metabolic intermediate-precursor serves not only as a major carbon source for most biosynthetic pathways that are essential for the growth and rapid proliferation of tumors, but also drives ATP synthesis during its catabolism to lactate. Under aerobic conditions more than half of the ATP produced in such tumor cells is derived via glycolysis, in sharp contrast to normal cells, where this value is usually less than 10% and oxidative phosphorylation is the predominant method for ATP generation. Under hypoxic (low oxygen tension) conditions, frequently present within tumors, the already high glycolytic rate may double, allowing the tumor cells to thrive while neighboring normal cells become growth deficient.

The important requirement of mitochondrially bound hexokinase for the high glycolytic phenotype of many tumors was demonstrated in experiments where galactose, which bypasses the hexokinase reaction in entering glycolysis, was substituted for glucose, and in experiments where tumor mitochondria were reconstituted with tumor cytosol. In the former experiments, the galactose for glucose substitution markedly lowered the glycolytic rate, and in the latter experiments the addition of tumor mitochondria, which contain high amounts of bound hexokinase (Type II), increased the low glycolytic capacity of tumor cytosol to the markedly elevated capacity observed in intact tumor cells. It has been shown also, that enhanced rates of glucose utilization and enhanced activities of membrane bound hexokinase are early events during cellular transformation of chicken embryo fibroblasts using a temperature sensitive Rous sarcoma virus mutant (Ts-68), where at permissive temperatures, an increase in glycolytic flux and an increase in hexokinase activity is observed along with cellular transformation.

Hexokinases Type I-III share two common properties; they have molecular weights of approximately 100 kDa and are sensitive to feedback inhibition by the product glucose-6-PO₄. In contrast, Type IV hexokinase (glucokinase) has a molecular weight of approximately 50 kDa and is insensitive to inhibition by physiologically relevant concentrations of glucose-6-PO₄. As such, mammalian glucokinase resembles hexokinases of yeast, which are insensitive to inhibition by glucose-6-PO4, and have molecular weights near 50 kDa. These observations implicate an evolutionary relationship, where the 100 kDa Types I-III mammalian hexokinases evolved from an ancestral 50 kDa enzyme similar to the yeast enzyme via a gene duplication and fusion event. In some such schemes, it is suggested that one of the duplicated catalytic sites (assigned to the N-terminal half of the 100 kDa enzyme) evolved to take on a regulatory function by acquiring the preferential capacity to bind glucose-6-PO₄. These views have gained support from evidence gathered at the genetic level during the last decade where the cDNA corresponding to each hexokinase has been cloned and sequenced and the exon-intron structure mapped, at least for the Type II hexokinase. Based on the cDNA data, and the deduced primary sequence (19,23-25), each of the two 50 kDa halves of the Type I-III isozymes, denoted N-half (about amino acids 1 to 465 in Type II) and C-half (about amino acids 466 to 917 in Type II), show close homology to each other, and to the 50 kDa hexokinase isozymes of yeast.

The proposed roles i.e., catalytic and regulatory that have been assigned to each C-and N-terminal half may require modification based on other data. Thus, site directed mutagenesis studies on recombinant Type I hexokinase show that both the regulatory properties (by glucose-6-PO₄) and catalytic functions reside within the C-terminal half of the enzyme, while the function of the N-terminal half remains to be elucidated. In contrast, recent studies on the Type II hexokinase have indicated that both the N- and C-terminal halves have similar catalytic properties. Therefore, at least for the functional aspects of mammalian hexokinases the proposed evolutionary views may require some modification. In any event, the hexokinase form (Type II) that is over-expressed by highly malignant tumors is the form in which both halves are active in converting glucose to the key metabolic precursor glucose-6-PO4.

Using somatic cell hybrids and in situ hybridization, various groups have mapped the chromosomal loci of hexokinase genes of both rat and human genomes. In rat, hexokinase Types I, II, III, and IV (glucokinase) have been assigned to chromosome bands 20q11, 4q34, 17q12, and 14q21, respectively. In humans, glucokinase has been mapped to 7p22, and Type I hexokinase to the short (10p11) or long arm (10q11) of chromosome 10. Type II hexokinase has been mapped to the chromosome 2p13 locus and Type III hexokinase to 5q35. Thus, in both rats and humans each of the hexokinase genes, including the tumor-related Type II gene resides on a separate chromosome.

The terms “polynucleotide” and “oligonucleotide” refer to molecules formed from joined nucleotides. The terms “polynucleotide” (and “oligonucleotides”) include naturally occurring polynucleotides or synthetic polynucleotides formed from naturally occurring subunits or analogous subunits designed to confer special properties on the polynucleotide so that it is more stable in biological systems or binds more tightly to target sequences. It also includes modifications of the polynucleotides such as chemically linking them to other compounds that will enhance delivery to cells or to the nucleus and other compartments of cells. Further, polynucleotides of the invention may contain modified internucleotide linkages to provide stability against nucleases. For example, the invention may include phosphorothioate oligodeoxyribonucleotides. Thus, the term “polynucleotide” includes unmodified multimers of ribonucleotides and/or deoxyribonucleotides, as well as multimers wherein one or more purine or pyrimidine moieties, sugar moieties or internucleotide linkages is chemically modified.

For example, as used herein the term “polynucleotide” and “oligonucleotide” as used herein include polymers of ribonucleic acid (RNA) or deoxyribonucleic acid (DNA) or mimetic thereof. They include multimers of natural or modified monomers or linkages capable of specifically binding to a target polynucleotide by way of a regular pattern of monomer-to-monomer interactions, such as Watson-Crick type of base pairing, Hoogsteen or reverse Hoogsteen types of base pairing, or the like. The terms “polynucleotide” and “oligonucleotide” include polynucleotides and oligonucleotides composed of naturally-occurring nucleobases, sugars and covalent internucleoside (backbone) linkages as well as polynucleotides and oligonucleotides having non-naturally-occurring portions which function similarly. Such modified or substituted polynucleotides and oligonucleotides are often preferred over native forms because of desirable properties such as, for example, enhanced cellular uptake, enhanced affinity for nucleic acid target and increased stability in the presence of nucleases.

While antisense polynucleotides and oligonucleotides are a preferred form of antisense compound, the present invention comprehends other polymeric and oligomeric antisense compounds, including but not limited to oligonucleotide mimetics such as are described below. The antisense compounds in accordance with this invention preferably comprise from about 100 to 3000 nucleobases. As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include polynucleotides containing modified backbones or non-natural intemucleoside linkages. As defined in this specification, polynucleotides having modified backbones include those that retain a phosphorus atom in the backbone and those that do not have a phosphorus atom in the backbone. For the purposes of this specification, and as sometimes referenced in the art, modified polynucleotides that do not have a phosphorus atom in their internucleoside backbone can also be considered to be polynucleosides.

Preferred modified polynucleotide and oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalklyphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,625,050; and 5,697,248, each of which is herein incorporated by reference.

Preferred modified oligonucleotide backbones that do not include a phosphorus atom therein have backbones that are formed by short chain alkyl or cycloalkyl intemucleoside linkages, mixed heteroatom and alkyl or cycloalkyl intemucleoside linkages, or one or more short chain heteroatomic or heterocyclic intemucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other preferred polynucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science, 254, 1497 (1991).

Most preferred embodiments of the invention are polynucleotides with phosphorothioate backbones and polynucleosides with heteroatom backbones, and in particular —CH₂—NH—O—CH₂—, —CH₂—N(CH₃)—O—CH₂—(known as a methylene (methylimino) or MMI backbone), —CH₂—O—N(CH₃)—CH₂—, —CH₂—N(CH₃)—N(CH₃) —CH₂—and—O—N(CH₃)—CH.₂—CH₂— (wherein the native phosphodiester backbone is represented as —O—P—O—CH₂—) of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH, F, O—, S—, or N-alkyl, O—, S—, or N-alkenyl, or O—, S— or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙ O]sub CH₃, O(CH₂)ₙ OCH₃, O(CH₂)ₙ NH2, O(CH₂)ₙ CH₃, O(CH₂)ₙ ONH2, and O(CH₂)ₙ ON[(CH₂)ₙ CH₃)]₂, where n and m are from 1 to about 10. Preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′—O—CH₂ CH₂ OCH₃, also known as 2′-O-(2-methoxyethyl) or 2′-MOE) (Martin et al., Helv. Chim. Acta, 78, 486 (1995)) i.e., an alkoxyalkoxy group. A further preferred modification includes 2′-dimethylaminooxyethoxy, i.e., a 2′-O(CH₂)₂ ON(CH₃)₂ group, also known as 2′-DMAOE, as described in U.S. Pat. No. 6,127,533, the contents of which are herein incorporated by reference.

Other preferred modifications include 2′-methoxy (2′—O—CH₃), 2′-aminopropoxy (2′—OCH₂ CH₂ CH₂ NH₂) and 2′-fluoro (2′—F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugars structures include, but are not limited to, U.S. Pat. Nos.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,0531 5,639,873; 5,646,265; 5,658,873; 5,670,633; and 5,700,920, each of which is herein incorporated by reference.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7- deazaadenine and 3-deazaguanine and 3-deazaadenine. Further nucleobases include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, those disclosed by Englisch et al., Angewandte Chemie, International Edition, 30, 613 (1991), and those disclosed by Sanghvi, Y. S., Chapter 15, Antisense Research and Applications, pages 289-302, Crooke, S. T. and Lebleu, B., ed., CRC Press, 3. Certain of these nucleobases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos.: 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, each of which is herein incorporated by reference.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 86, 6553 (1989)), cholic acid (Manoharan et al., Bioorg. Med. Chem. Lett., 4, 1053 (1994)), a thioether, e.g., hexyl-S-tritylthiol (Manoharan et al., Ann. N.Y. Acad. Sci., 660, 306 (1992)); Manoharan et al., Bioorg. Med. Chem. Let., 3, 2765 (1993)), a thiocholesterol (Oberhauser et al., Nucl. Acids Res., 20, 533 (1992), an aliphatic chain, e.g., dodecandiol or undecyl residues (Saison-Behmoaras et al., EMBO J., 10, 111 (1991); Kabanov et al., FEBS Lett., 259, 327 (1990); Svinarchuk et al., Biochimie, 75, 49 (1993)), a phospholipid, e.g., dihexadecyl-rac-glycerol or triethylammonium 1,2-di-O-hexadecyl-rac-glycero-3-H-phosphonate (Manoharan et al., Tetrahedron Lett., 36, 3651 (1995); Shea et al., Nucl. Acids Res., 18, 1990)), a polyamine or a polyethylene glycol chain (Manoharan et al., Nucleosides & Nucleotides, 14, 969 (1995)), or adamantane acetic acid (Manoharan et al., Tetrahedron Lett., 36, 3651 (1995)), a pahnityl moiety (Mishra et al., Biochim. Biophys. Acta, 1264, 229 (1995)), or an octadecylamine or hexylamino-carbonyl-oxycholesterol moiety (Crooke et al., J. Pharmacol. Exp. Ther., 277, 923(1996)).

Representative United States patents that teach the preparation of such oligonucleotide conjugates include, but are not limited to, U.S. Pat. Nos.: 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos.: 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. The antisense compounds of the invention are synthesized in vitro and do not include antisense compositions of biological origin. For example, Type II hexokinase cDNA can be prepared by polymerase chain reaction amplification (See Example for details). Polynucleotides can be prepared using synthetic chemical methods, such as, for example, phosphoramidite chemistry by sulfurization with tetraethylthiuram disulfide in acetonitrile. (See, for example, Vu and Hirschbein, Tetrahedron Lett. 1991, 32, 30005-30008.) Polynucleotides of the invention may also be synthesized using in vitro and in vivo transcription systems, such as transcription by T.sup.7 polymerase or expression vectors. Polynucleotides synthesized using in vitro and in vivo transcription systems may be modified via chemical methods known to those skilled in the art. Examples of such modifications include encapsulation in liposomes, or chemical linkage to steroids, antibodies, and cell receptor ligands.

The length of the polynucleotide moiety should be sufficiently large to ensure that specific binding will take place only at the desired target polynucleotide and not at other fortuitous sites, as explained in many references, e.g., Rosenberg et al., International Application PCT/US92/05305; or Szostak et al., Meth. Enzymol, 68:419-429 (1979). The upper range of the length is determined by several factors, including the inconvenience and expense of synthesizing and purifying large polymers, the greater tolerance of longer polynucleotides for mismatches than shorter polynucleotides, whether modifications to enhance binding or specificity are present, whether duplex or triplex binding is desired, and the like.

An “antisense polynucleotide that hybridizes with a mRNA encoding hexokinase” is a polynucleotide having a sequence capable of forming a stable duplex with a portion of an mRNA transcript of a hexokinase gene. “Stability” in reference to duplex formation roughly means how tightly an antisense polynucleotide binds to its intended target sequence; more precisely, it means the free energy of formation of the duplex or triplex under physiological conditions. Melting temperature under a standard set of conditions, as described herein, e.g., is a convenient measure of duplex and/or triplex stability. Preferably, antisense polynucleotides of the invention are selected that have melting temperatures of at least 50° C. under the standard conditions; thus, under physiological conditions and the preferred concentrations, duplex formation will be substantially favored over the state in which the antisense polynucleotide and its target are dissociated. It is understood that a stable duplex may include mismatches between base pairs. Preferably, antisense polynucleotides of the invention form perfectly matched duplexes with their target polynucleotides.

Mismatches can occur because of “conservative variation” in the nucleotide sequence encoding the polypeptide encoded by the target polynucleotide. As used herein, “conservative variation” and “substantially similar” means the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic acid for aspartic acid, or glutamine for asparagine, and the like. The terms “conservative variation” and “substantially similar” also include the use of a substituted amino acid in place of an unsubstituted parent amino acid provided that antibodies raised to the substituted polypeptide also immunoreact with the unsubstituted polypeptide.

Polynucleotides used in invention methods can be modified to increase stability and prevent intracellular and extracellular degradation. It is more preferred that the polynucleotides of the invention be modified to increase their affinity for target sequences, and their transport to the appropriate cells and cell compartments when they are delivered into a mammal in a pharmaceutically active form.

In general, the antisense polynucleotides used in the practice of the present invention will have a sequence which is complementary to at least a portion of the target polynucleotide. Absolute complementarity is not required. Thus, reference herein to a “nucleotide sequence complementary to” a target polynucleotide does not necessarily mean a sequence having 100% complementarity with the target segment. In general, any polynucleotide having sufficient complementarity to form a stable duplex with the target (e.g. hexokinase mRNA) that is, a polynucleotide which is “hybridizable”, is suitable. Stable duplex formation depends on the sequence and length of the hybridizing polynucleotide and the degree of complementarity with the target polynucleotide. Generally, the larger the hybridizing polymer (or oligomer), the more mismatches may be tolerated. One skilled in the art may readily determine the degree of mismatching which may be tolerated between any given antisense oligomer and the target sequence, based upon the melting point, and therefore the thermal stability, of the resulting duplex. The thermal stability of hybrids formed by the antisense poynucleotides used in invention methods are determined by way of melting, or strand dissociation, curves. The temperature of fifty percent strand dissociation is taken as the melting temperature, Tₘ, which, in turn, provides a convenient measure of stability. Tₘ measurements are typically carried out in a saline solution at neutral pH with target and antisense polynucleotide concentrations at between about 1.0-2.0 μM. Typical conditions are as follows: 150 mM NaCl and 10 mM MgCl₂ in a 10 mM sodium phosphate buffer (pH 7.0) or in a 10 mM Tris-HCl buffer (pH 7.0). Data for melting curves are accumulated by heating a sample of the antisense polynucleotide/target polynucleotide complex from room temperature to about 85-90° C. As the temperature of the sample increases, absorbance light at 260 nm is monitored at 1° C. intervals, e.g., using a Hewlett-Packard (Palo Alto, Calif.) model HP 8459 UV/VIS spectrophotometer and model HP 89100A temperature controller, or like instruments. Such techniques provide a convenient means for measuring and comparing the binding strengths of antisense polynucleotides of different lengths and compositions.

Recombinant nucleic acid molecules comprising a polynucleotide complementary to the nucleotide sequence encoding hexokinase includes vectors containing antisense nucleic acid, ribozymes, or triplex agents to block transcription or translation of a hexokinase mRNA, either by masking that mRNA with an antisense nucleic acid or triplex agent, or by cleaving it with a ribozyme in disorders associated with increased hexokinase expression.

Use of a polynucleotide to stall transcription is known as the triplex strategy since the oligomer winds around double-helical DNA, forming a three-strand helix. Therefore, these triplex compounds can be designed to recognize a unique site on a chosen gene (Maher et al., (1991) Antisense Res. and Dev., 1:227; Helene, (1991) Anticancer Drug Design, 6:569).

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it (Cech, 1988, J Amer. Med. Assn., 260:3030). A major advantage of this approach is that, because they are sequence-specific, only mRNAs with particular sequences are inactivated.

There are two basic types of ribozymes namely, tetrahymena-type (Hasselhoff (1988) Nature, 334:585) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-based recognition sequences are preferable to shorter recognition sequences.

In preferred embodiments, antisense polynucleotides hybridize with Type II hexokinase mRNA or Type I hexokinase mRNA. In additional preferred embodiments, the mRNA has a nucleotide sequence as set forth in SEQ ID NO: 1 or SEQ ID NO:2.

For therapeutic or prophylactic treatment, polynucleotides are administered in accordance with this invention. Polynucleotides may be formulated in a pharmaceutical composition, which may include carriers, thickeners, diluents, buffers, preservatives, surface active agents and the like in addition to the polynucleotide. The polynucleotide may be administered in conjunction with other therapeutics found effective to inhibit or prevent proliferation of tumor cells.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents. The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66:1). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For polynucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid, and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine, and the like.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, continuous infusion, subcutaneous, intraperitoneal or intramuscular injection, pulmonary administration, e.g., by inhalation or intrathecal or intraventricular administration. For oral administration, it has been found that oligonucleotides with at least one 2′-substituted ribonucleotide are particularly useful because of their absorption and distribution characteristics. Oligonucleotides with at least one 2′-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions and/or formulations comprising the oligonucleotides of the present invention may also include penetration enhancers in order to enhance the alimentary delivery of the oligonucleotides. Penetration enhancers may be classified as belonging to one of five broad categories, i.e., fatty acids, bile salts, chelating agents, surfactants and non-surfactants (Lee et al., Critical Reviews in Therapeutic Drug Carrier Systems, 8, 91 (1991); Muranishi, Critical Reviews in Therapeutic Drug Carrier Systems, 7, 1 (1990)). One or more penetration enhancers from one or more of these broad categories may be included. Penetration enhancers are described in U.S. Pat. No. 6,083,923, which is herein incorporated by reference.

Various fatty acids and their derivatives which act as penetration enhancers include, for example, oleic acid, lauric acid, capric acid, myristic acid, palmitic acid, stearic acid, linoleic acid, linolenic acid, dicaprate, tricaprate, recinleate, monoolein (1-monooleoyl-rac-glycerol), dilaurin, caprylic acid, arachidonic acid, glyceryl 1-monocaprate, 1-dodecylazacycloheptan-2-one, acylcarnitines, acylcholines, mono-and di-glycerides and physiologically acceptable salts thereof (i.e., oleate, laurate, caprate, myristate, palmitate, stearate, linoleate, etc.) (Lee et al., Critical Reviews in Therapeutic Drug Carrier Systems, 1991, page 92; Muranishi, Critical Reviews in Therapeutic Drug Carrier Systems, 7, 1 (1990); El-Hariri et al., J. Pharm. Pharmacol. 44, 651 (1992). Examples of some presently preferred fatty acids are sodium caprate and sodium laurate, used singly or in combination at concentrations of 0.5 to 5%.

The physiological roles of bile include the facilitation of dispersion and absorption of lipids and fat-soluble vitamins (Brunton, Chapter 38 In: Goodman & Gilman's The Pharmacological Basis of Therapeutics, 9th Ed., Hardman et al., eds., McGraw-Hill, New York, N.Y., 1996, pages 934-935). Various natural bile salts, and their synthetic derivatives, act as penetration enhancers. Thus, the term “bile salt” includes any of the naturally occurring components of bile as well as any of their synthetic derivatives. A presently preferred bile salt is chenodeoxycholic acid, sodium salt (CDCA)(Sigma Chemical Company, St. Louis, Mo.), generally used at concentrations of 0.5 to 2%.

Complex formulations comprising one or more penetration enhancers may be used. For example, bile salts may be used in combination with fatty acids to make complex formulations. Preferred combinations include CDCA combined with sodium caprate or sodium laurate (generally 0.5 to 5%).

Chelating agents include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, 1991, page 92; Muranishi, Critical Reviews in Therapeutic Drug Carrier Systems, 7, 1 (1990); Buur et al., J. Control Rel., 14 (1990)). Chelating agents have the added advantage of also serving as DNase inhibitors.

Surfactants include, for example, sodium lauryl sulfate, polyoxyethylene-9-lauryl ether and polyoxyethylene-20-cetyl ether (Lee et al., Critical Reviews in Therapeutic Drug Carrier Systems, 1991, page 92); and perfluorochemical emulsions, such as FC-43 (Takahashi et al., J. Pharm. Phamacol., 1988, 40:252).

Non-surfactants include, for example, unsaturated cyclic ureas, 1 -alkyl- and 1 -alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39:621).

As used herein, “carrier compound” refers to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The co-administration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioated oligonucleotide in hepatic tissue is reduced when it is coadministered with polyinosinic acid, dextran sulfate, polycytidic acid or 4-acetamido-4′-isothiocyano-stilbene-2,2′-disulfonic acid (Miyao et al., Antisense Res. Dev., 5, 115 (1995); Takakura et al., Antisense & Nucl. Acid Drug Dev., 6, 177 (1996)).

In contrast to a carrier compound, a “pharmaceutically acceptable carrier” (excipient) is a pharmaceutically acceptable solvent, suspending agent or any other pharmacologically inert vehicle for delivering one or more nucleic acids to an animal. The pharmaceutically acceptable carrier may be liquid or solid and is selected with the planned manner of administration in mind so as to provide for the desired bulk, consistency, etc., when combined with a nucleic acid and the other components of a given pharmaceutical composition. Typical pharmaceutically acceptable carriers include, but are not limited to, binding agents (e.g., pregelatinized maize starch, polyvinyl-pyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.). Sustained release oral delivery systems and/or enteric coatings for orally administered dosage forms are described in U.S. Pat. Nos. 4,704,295; 4,556,552; 4,309,406; and 4,309,404.

The compositions of the present invention may additionally contain other adjunct components conventionally found in pharmaceutical compositions, at their art-established usage levels. Thus, for example, the compositions may contain additional compatible pharmaceutically-active materials such as, e.g., antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the invention.

Regardless of the method by which the antisense compounds of the invention are introduced into a patient, colloidal dispersion systems may be used as delivery vehicles to enhance the in vivo stability of the compounds and/or to target the compounds to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid:oligonucleotide complexes of uncharacterized structure. A preferred colloidal dispersion system is a plurality of liposomes. Liposomes are microscopic spheres having an aqueous core surrounded by one or more outer layer(s) made up of lipids arranged in a bilayer configuration (see, generally, Chonn et al., Current Op. Biotech., 1995, 6, 698). An example of liposome preparation is described in U.S. Pat. No. 6,083,923, which is herein incorporated by reference.

The antisense compounds of the invention may be combined with (a) liposomes; (b) one or more antisense compounds and (c) one or more other chemotherapeutic agents which function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine, 5-fluorouracil (5-FU), floxuridine (5-FUdR), methotrexate, colchicine, vincristine, vinblastine, etoposide, teniposide, cisplatin and diethylstilbestrol (DES). (See, generally, The Merck Manual of Diagnosis and Therapy, 15th Ed., Berkow et al., eds., 1987, Rahway, N.J., pages 1206-1228)). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. (See, generally, The Merck Manual of Diagnosis and Therapy, 15th Ed., Berkow et al., eds., 1987, Rahway, N.J., pages 2499-2506 and 46-49, respectively)). Other non-antisense chemotherapeutic agents are also within the scope of this invention. Two or more combined compounds may be used together or sequentially.

The present invention also provides a pharmaceutical preparation useful for inhibiting proliferation of tumor cells having a highly glycolytic phenotype in vivo comprising an antisense oligonucleotide that hybridzes with a mRNA encoding hexokinase.

Also provided is a recombinant nucleic acid vector comprising five elements operably linked in a 5′ to 3′ direction. The five elements are: (a) a 5′ long terminal repeat (LTR) sequence; (b) a multiple cloning site (MCS) for restriction enzymes EcoRI, HpaI, XhoI, and BamHI; (c) an internal ribosome entry sequence (IRES); (d) a selectable marker sequence; and (e) a 3′ long terminal repeat (LTR) sequence.

The vector can be a cloning vector, which is useful for maintaining the polynucleotide, or can be an expression vector, which contains, in addition to the polynucleotide, regulatory elements useful for expressing the polynucleotide and, where the polynucleotide encodes a peptide, for expressing the encoded peptide in a particular cell. An expression vector can contain the expression elements necessary to achieve, for example, sustained transcription of the encoding polynucleotide, or the regulatory elements can be operatively linked to the polynucleotide prior to its being cloned into the vector.

An expression vector (or the polynucleotide) generally contains or encodes a transcriptional regulatory element, which can provide constitutive or, if desired, inducible or tissue specific or developmental stage specific expression of the encoding polynucleotide, a poly-A recognition sequence, and a ribosome recognition site or internal ribosome entry site, or other regulatory elements such as an enhancer, which can be tissue specific. As used herein, the term “transcriptional regulatory oligonucleotide” or “transcriptional regulatory element” or the like, refers to a nucleotide sequence that can effect the level of transcription of an operatively linked polynucleotide. Thus, the term encompasses oligonucleotide sequences that increase the level of transcription of a polynucleotide, for example, a promoter element or an enhancer element, or that decrease the level of transcription of a polynucleotide, for example, a silencer element. A transcriptional regulatory element can be constitutively active or inducible, which can be inducible from an inactive state or from a basal state, and can be tissue specific or developmental stage specific, or the like.

The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J Bioenerg. Biomemb. 25:37-42, 1993; Kirshenbaum et al., J. Clin. Invest. 92:381-387, 1993; each of which is incorporated herein by reference).

As used herein, the term “multiple cloning site” or “MCS” refers to a nucleic acid sequence containing two or more restriction enzyme sites. For example, a nucleic acid sequence can encode sites for the restriction enzymes EcoRI, HpaI, XhoI, and BamHI.

As used herein, the term “internal ribosome entry site” or “IRES” refers to a site that recurits ribosomes allowing a mammalian expression system to act in a polycistronic manner, i.e., one single mRNA transcript can encode several genes. Generally, mammalian systems are monocistroic while bacterial systems are generally polycistronic. By placing an IRES between a cDNA of interest, for example, a cDNA encoding hexokinase, and a cDNA encoding a selectable marker, for example, neomycin, the expression of antisense hexokinase can be linked to the amount of marker, e.g., antibiotic, used to select the transfected cells. Cells that survive at high concentrations of antibiotic will express high amounts of the nucleic acid sequence encoded by the cDNA of interest, e.g., hexokinase antisense RNA (see Examples section).

As used herein, the term “operatively linked” means that two or more molecules are positioned with respect to each other such that they act as a single unit and effect a function attributable to one or both molecules or a combination thereof. For example, two polynucleotide sequences, each encoding a protein, Type II hexokinase and neomycin phosphotransferase, for example, can be operatively linked via a regulatory element, IRES, for example, in which case the regulatory element confers its regulatory effect on both polynucleotides similarly to the way in which the regulatory element would effect a polynucleotide sequence with which it normally is associated with in a cell. The mRNA can contain an internal ribosome entry site (IRES) sequence, which effects the manner in which ribosomes bind to an mRNA and initiate translation, and does not require interaction of the ribosome with the 5′ end of an mRNA transcript. Thus, an IRES element can confer an additional level of regulation of gene expression by allowing for direct correlation between a selectable marker gene (e.g., neomycin phosphotransferase), and an expressed gene of interest (e.g., hexokinase) either in ‘sense or antisense’ orientations.

In a preferred embodiment of the invention, the vector is a modified pLXSN vector (Clontech Laboratories, Inc., Palo Alto, Calif.). The unmodified vector contains elements derived from Moloney murine leukemia virus (MoMuLV) and Moloney murine sarcoma virus (MoMuSV), and is designed for retroviral gene delivery and expression. Upon transfection into a packaging cell line, pLXSN can transiently express, or integrate and stably express, a transcript containing Ψ⁺ (the extended viral packaging signal), the gene of interest, and a selectable marker. The 5′ viral LTR in this vector contains promoter/enhancer sequences that control expression of the gene of interest in the multiple cloning site. The SV40 early promoter (P_{SV40e}) controls expression of the neomycin resistance gene (Neo^{r}), which allows antibiotic selection in eukaryotic cells. pLXSN also includes the Col El origin of replication and E. coli Amp^{r} gene for propagation and antibiotic selection in bacteria.

A selectable marker sequence is a nucleic acid sequence, the presence of which allows one to detect the presence of at least one additional nucleic acid sequence that is linked to the selector marker sequence. The marker gene may be inserted in the gene to be identified, thereby marking the presence of the recombinant nucleic acid construct, while providing a positive selectable marker. A preferred selectable maker sequence is a sequence that confers resistance to an antibiotic. For example, a sequence encoding neomycin phosphotransferase confers neomycin resistance.

Once in the cell, RNA from the vector is packaged into infectious, replication- incompetent retroviral particles. Subsequent introduction of pLXSN, containing Ψ⁺(psi), transcription and processing elements, and the gene of interest produces high- titer, replication-incompetent infectious virus. These retroviral particles can therefore infect target cells and transmit the gene of interest (which is cloned between the viral LTR sequences), but cannot replicate within these cells since the cells lack the viral structural genes.

A vector may be modified by removal and/or addition of nucleic acid sequences. Removal of certain sequences can be accomplished by restriction enzyme digestion at suitable sites known to those of skill in the art. For example, nucleic acids encoding the SV40 promoter can be removed by digestion from vector pLXSN (Genbank Accession Number M28248). Nucleic acids encoding an IRES sequence of encephalomyocarditis virus can be added to the same vector, (see Examples section) thereby conferring the properties of the IRES sequence on the pLXRN vector. During such replacement, original restriction sites of the pLXSN vector, EcoRI, HpaI, XhoI, and BamHI can be retained. The complete sequence of modified cloning vector pLXRN is set forth in SEQ ID NO:3, and provided in Accession No. AF113968 A vector can be modified by the addition of an antisense polynucleotide. Provided herein is a vector that includes a polynucleotide having the nucleotide sequence complementary to the sequence set forth in SEQ ID NO: 1 (“antisense polynucletide”). The antisense polynucleotide can be located 5′ of a regulatory sequence, for example 5′ of an IRES sequence.

The present invention also provides a recombinant nucleic acid vector comprising a polynucleotide complementary to the nucleotide sequence encoding Type II hexokinase (SEQ ID NO: 1). Also provided is a recombinant nucleic acid vector comprising a polynucleotide complementary to the nucleotide sequence encoding Type I hexokinase (SEQ ID NO:2).

The antisense compounds of the present invention can be utilized for diagnostics, therapeutics, prophylaxis and as research reagents and kits. For therapeutics, an animal, preferably a human, suspected of having a disease or disorder which can be treated by modulating the expression of a hexokinase is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

The antisense compounds of the invention are useful for research and diagnostics, because these compounds hybridize to nucleic acids encoding hexokinase, enabling sandwich and other assays to easily be constructed to exploit this fact. Hybridization of the antisense oligonucleotides of the invention with a nucleic acid encoding hexokinase can be detected by means known in the art. Such means may include conjugation of an enzyme to the oligonucleotide, radiolabelling of the oligonucleotide or any other suitable detection means. Kits using such detection means for detecting the level of a hexokinse in a sample may also be prepared.

The following examples are intended to illustrate but not limit the invention.

### EXAMPLE 1

To test the usefulness of hexokinase as a target in therapeutic treatment of tumors, the highly glycolytic, rapidly growing hepatoma cell line AS-30D was used. Of the four hexokinase isozymes (Mathupala et al. (1997) J. Bioenerg. Biomemb. 29: 339-343) (Types I, II, III and IV), it is the Type II, and to a lesser extent Type I that are over-expressed in such rapidly growing, highly glycolytic tumors (Nakashima et al (1984) Cancer Res. 44: 5702-5706; Shinohara et al.. (1991) FEBS Lett. 291: 55-57; Rempel et al. (1994) Biochim. Biophys. Acta 1219:60-668; Mathupala et al. (1995) J. Biol. Chem. 270: 16918-16925) The studies outlined here were designed to test methods to selectively silence both Type II and Type I hexokinase gene expression and inhibit glycolysis to test whether this will arrest or slow tumor growth. The experiments were carried out to target the hexokinase transcripts by using anti-sense RNA techniques (Rees et al. (1996) Biotechniques 20: 102-110).

Materials

First Strand cDNA Synthesis System was from Life Technologies. RNA was isolated using a modified guanidium thiocyanate-phenol system (RNA Stat-60, Tel-test, Inc. Texas). Retroviral vector pLXSN (Nakashima et al. (1988) Cancer Res. 44: 5702-5706) was a kind gift from Dr. Dusty Miller of Fred-Hutchinson Cancer Center, Seattle, Wash. Vector pIRES1Neo (Barnes, W. M. (1994) Proc. Natl. Acad. Sci. USA 91:2216-2220) was from Clonetch, California. Restriction enzymes, and DNA modifying enzymes, tissue culture media and other media components were from Life Technologies, Maryland or from Sigma Chemical Co, Missouri. FBS was from Atlanta Biologicals. [6-³H] thymidine (1 mCᵢ/ml; 19.2 Ci/mmol) was from DuPont NEN.

Tumor Cells

AS-30D hepatoma cells, a model tumor cell line exhibiting high glycolytic rates (Chomczynski and Sacchi (1987) Anal. Biochem. 162: 156-159) were propagated in the peritoneal cavity of female Sprague-Dawley rats (100-150 g) and isolated as described previously using glucose supplemented RPMI-1640 media, in the absence of serum (Miller and Rosman (1989) Biotechniques 7: 980-990). These were re-suspended in a final concentration of 50×10⁶ cells m⁻¹ for further studies. A thermostable DNA polymerase mix of Taq polymerase and Pfu polymerase (Taq Extender PCR additive, Stratagene) (Thelenand Wilson. (1991) Arch. Biochem. Biophys. 286:645-651) was used in all PCR amplification reactions described herein.

Cloning of the Type II Hexokinase CDNA

Total RNA was isolated from purified AS-30D hepatoma cells using a modified acid guanidium thiocyanate-phenol-chloroform (Miller et al .(1993) Meth. Enzymology 217: 581-599) (RNA Stat-60) according to manufacturer's protocol. 5 μg RNA (in 1 mM EDTA, pH 7.0) was primed with random primers and first strand cDNA synthesis carried out using Superscript II reverse-transcriptase (Life Technologies). The coding region of type II hexokinase cDNA was amplified from the first strand synthesis mix by PCR. In brief, two PCR primers that contained the start and stop codons of normal rat muscle type II hexokinase (Freshney, R. I. in Culture of Animal Cells: A Manual of basic technique, Second Ed. Wiley-Liss, New York) that incorporated an EcoRI and an Xba I site at their respective 5′ termini (forward primer, 5′-GGA-ATT-CAT-GAT-CGC-CTC-GCA-TAT-GAT-C-3′ (SEQ ID NO:4) ; reverse primer, 5′-GCT-CTA-GAC-TAT-CTC-TGC-CCA-GCC-TCC-CG-3′ (SEQ ID NO:5)) were used to amplify the type II hexokinase cDNA. 40 cycle PCR was carried out using the parameters 94° C., 5 min, 55° C., 2 min, 72° C., 3 min (1 cycle); 94° C., 1 min, 55° C., 2 min, 72° C., 3 min (40 cycles); 94° C., 1 min, 55° C., 2 min, 72° C., 10 min (1 cycle). The single-band PCR product was purified by ion-exchange spin chromatography (Prep-a-gene, Bio-Rad), double-digested with EcoRI and XbaI. The cDNA was purified by agarose gel electrophoresis (0.8%) to remove the linker termini, ligated into pUC 18 vector at the same restriction sites and used to transform E. coli SURE (Stratagene). Two transformants were selected and sequenced in both orientation to deduce the sequence of type II hexokinase cDNA of hepatoma.

Construction of the pLXRN bicistronic vector

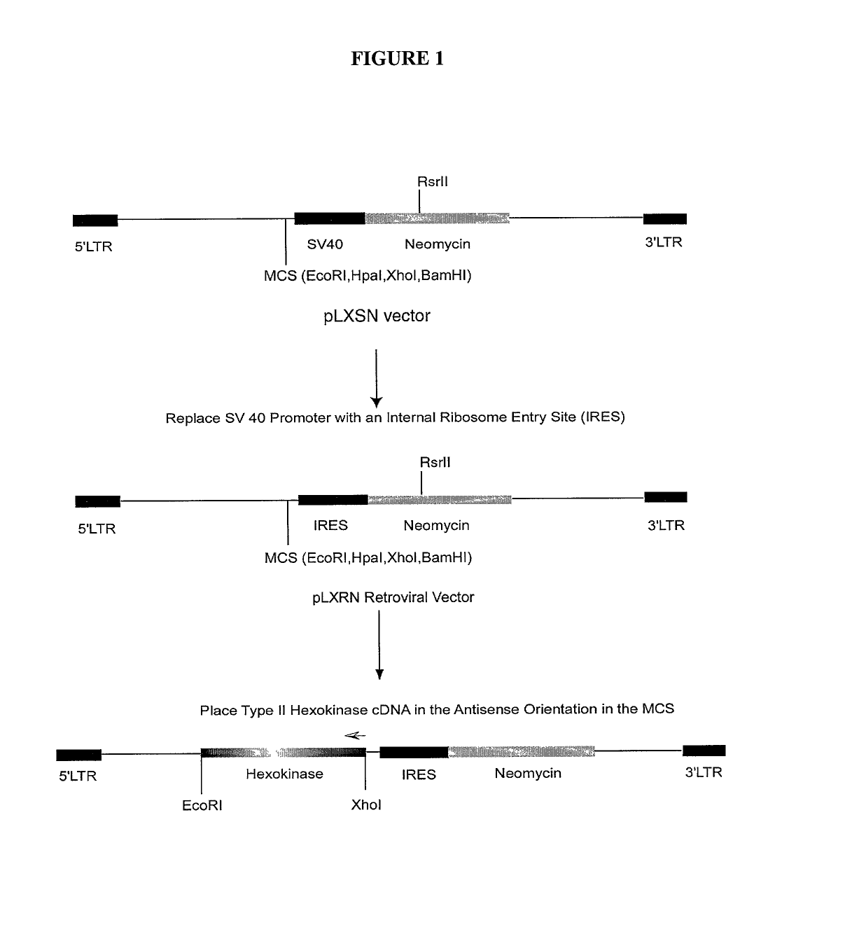

A DNA segment corresponding to the SV40 promoter and part of the neomycin phosphotransferase cDNA of pLXSN vector was removed by double-digestion with Bam HI and RsrII (FIG. 1). The deleted region was replaced with an internal ribosome entry sequence (IRES) to generate a bicistronic retroviral vector for the study as follows. The IRES sequence, along with the neomycin phosphotransferase cDNA segment that corresponds to the same region removed from the pLXSN vector (see above), were PCR amplified from pIRES1Neo vector (Clontech). The primers contained a BamHI site or an RsrII site at their respective termini (forward primer, 5′-CGG-ATC-CGC-CCC-TCT-CCC-TCC-C-3′ (SEQ ID NO:6); reverse primer, 5′-GAT-AGC-GGT-CCG-CCA-CAC-CCA-GCC-3′ (SEQ ID NO:7)) for exact positional cloning. The PCR product was purified by ion-exchange spin chromatography as before, and double digested with Bam HI and RsrII. This was agarose gel purified and cloned into the same sites of the pLXSN vector backbone to generate the bicistronic retroviral vector pLXRN. The vector was propagated in E. coli DH5α and the ligation sites sequenced in both orientations to verify cloning integrity.

Construction of the type II hexokinase-antisense vector

Type II hexokinase cDNA was amplified from the earlier pUC 18 clone using two primers with an XhoI site and an EcoRI site at their respective 5′ termini (forward primer, 5′-GCC-TCG-AGC-CTC-GGT-TTC-TCT-ACT-C-3′ (SEQ ID NO:8); reverse primer, 5′-GGA-ATT-CCT-ATC-TCT-GCC-CAG-CCT-CCC-G-3′(SEQ ID NO:9)). 30 cycle PCR was carried out using the parameters 94° C., 5 min, 50° C., 5 min, 72° C., 3 min (1 cycle); 94° C., 1 min, 50° C., 1 min, 72° C., 3 min (30 cycles); 94° C., 1 min, 50° C., 1 min, 72° C., 10 min (1 cycle). PCR product was digested, purified as described before, and cloned in anti-sense orientation into the EcoRI, XhoI sites of the MCS of pLXRN vector.

Production of Retrovirus

Non-replicative retrovirus were produced in the ecotropic packaging cell line PE501 according to established protocols (Thompson and Ioli (1997) Biotechniques 22: 613-614) using both pLXRN parental vector and the pLXRN-antisense hexokinase constructs.

Adaptation of AS-30D cells for growth in glucose deficient media

In order to carry out the antisense experiments, the AS-30D cells were adapted to glucose deficient media as follows; Freshly isolated AS-30D cells were plated onto poly-L-lysine coated (Baijal and Wilson (1995) Arch. Bioche. Biophys. 321:413-420) tissue culture vessels (10⁶ cells per 100 mm² plate) using Leibovitz's L-15 media supplemented with 1.2 mg/L NaHCO₃ and 3.6 g/L HEPES, and 10% FBS for growth in the presence of 5% CO₂. The cells were adapted for 2 weeks and then the media changed to a glucose deficient DMEM supplemented as follows; 1.2 g NaHCO₃/L, 15 mg/L phenol red, 6.0 g/L HEPES, 0.55 g/L sodium pyruvate, 4 mM glutamine, antibiotic/anti-mycotic mix, and 10% FBS. The cells were adapted to this media for 2 weeks and then exposed to recombinant retrovirus as follows.

Transduction of AS-30D cells and selection of permanent transfectants

5×10⁵ cells in 4.0 ml glucose deficient DMEM media (in the absence of FBS and antibiotics/antimycotics) were placed in poly-L-lysine coated 6-well multiwell plates and exposed to recombinant retrovirus at 100 m.o.i. according to standard protocols. After 24 hrs, the cells were trypsinized and plated at 10-fold dilution in 100 mm² tissue culture plates in fresh media supplemented with FBS and 725 μg/ml G-418 (geneticin). Media replacement was carried out once every 48 hrs for 2 weeks and clonal isolates ( about 100 cells per clone) recovered by a colony-lift procedure (Wilson (1995) Rev. Physiol. Biochem. Pharmacol. 126:65-198) and further propagated in 100 mm² plates for 2 weeks.

Hexokinase Assay

Sham-transfected (with virus generated with parental pLXRN vector) and antisense hexokinase transfected cells were lysed in situ using a TritonX- 100 lysis buffer and the lysate assayed for hexokinase activity as described (Rempel et al. (1996) . FEBS Lett. 385: 233-237). In brief, clonal isolates from sham-transfected (AS-30D hepatoma cells processed through the transfection and selection procedures, but in the absence of retrovirus and G-418 selection medium), control (AS-30D hepatoma cells transfected with the parent pLXRN retrovirus and selected in G-418 containing medium), and anti-sense (AS-30D hepatoma cells transfected with the recombinant pLXRN retrovirus harboring the Type II Hexokinase cDNA in anti-sense orientation and selected in G-418 medium) cells were grown to confluency (10 ×10⁶ cells) in the case of sham-transfected and control clonal isolates, or up to 5×10⁵ cells for anti-sense clonal isolate in 100 mm tissue culture dishes and then lysed in hexokinase lysis buffer (50 mM sodium phophate, 10 mM glucose, 10 mM thioglycerol, 0.1% Triton X-100, pH 7.5). The clear lysate was assayed for low Kₘ hexokinase activity (both Type I and Type II hexokinase) essentially as described by Parry and Pedersen (Parry and Pedersen (1983) J. Biol. Chem. 258:10904-10912, incorporated by reference herein). Total protein was assayed by the Bradford method (Bio-Rad Protein Assay).

Western Analysis

Cell extracts used for the hexokinase assay were separated on 7.5% SDS-polyacrylamide gels and then transferred onto PVDF (Bio-Rad) membranes in CAPS buffer (10 mM 3-cyclohexylamino-1-propanesulfonate, 10% v/v methanol, pH 11). The membranes were probed with a type II hexokinase polyclonal antibody, and detected by chemiluminescence (ECL System, Amersham, Mass.).

Proliferative Assay

The media of parallel clones of sham-transfected and antisense isolates were plated in 24-well multiwell plates (25×10³ cells per plate) in -quadruplicate, and the media replaced with DMEM supplemented with glucose. After for 24 hrs, the medium was replaced with the same containing 1 μCi/ml ³H-thymidine per well and incubated for 12 hrs. The cells were then washed in cold PBS, and precipitated with 10% TCA to remove unincorporated ³H-thymidine and acid soluble material. Acid insoluble material was dissolved in 0.5 ml lysis buffer (0.1% Triton X-100, 0.3 M NaOH), and 50 ml aliquots analyzed by scintillation counting.

### EXAMPLE 2

### Type II Hexokinase Is Essentially Identical In Both Normal And Tumor Cells

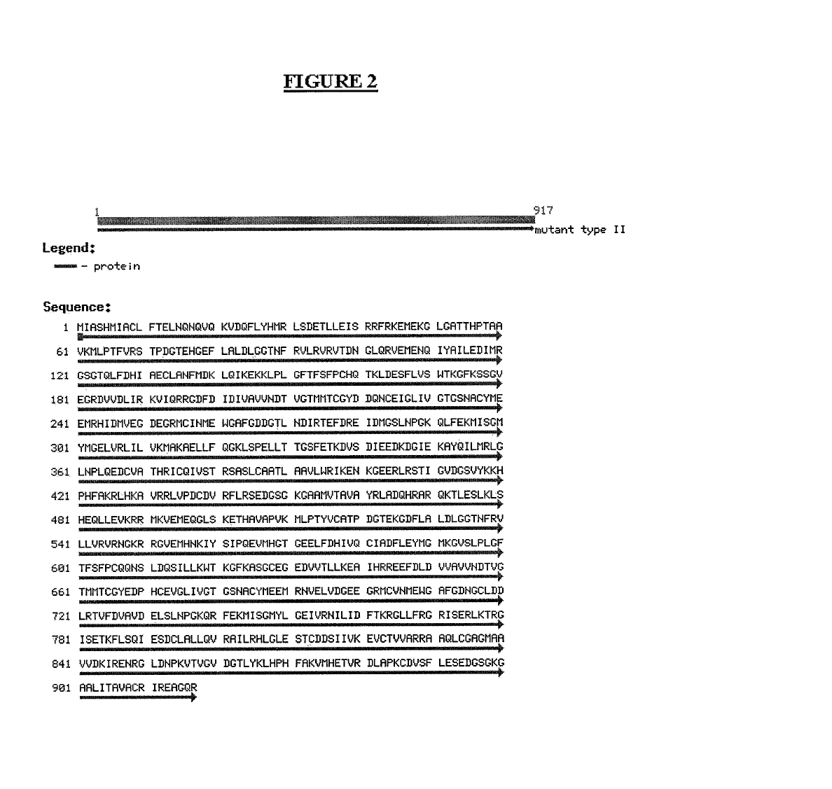

Analysis of the cloned sequence of type II hexokinase cDNA and comparison with the same isoform isolated from normal tissue indicated only four amino acid changes at the primary sequence level (Pro¹¹⁴-Leu¹¹⁴; Ala⁹²-Val^{192;} Phe⁷⁸²-Ser⁷⁸²; Leu⁸⁵⁴-Pro⁸⁵⁴) (FIG. 2). None of the changes are located in functionally important catalytic or regulatory regions. Therefore, the enhanced activity seen for this enzyme resides in enhanced transcription of the gene in highly glycolytic tumors as well as amplification of the gene.

Bicistronic retroviral vectors a provide a highly effective means of transgene expression: With the advent of bicistronic vector systems, it has been possible to rapidly select positive clones based on a direct correlation of selection marker gene expression to the expression of the transgene of interest. Here, a bicistronic retroviral vector was constricted by inserting an IRES (Jackson et al. (1990) Trends Biochem. Sci 15: 477-483) with a multiple cloning sequence identical to the EcoRI-HpaI-XhoI-BamHI MCS cassette that is common to most retroviral vectors used in gene therapy. Therefore, the engineered pLXRN vector can be used for direct transfer of transgenes of interest between the commonly used monocistronic vectors to the more advantageous bicistronic system as described herein.

### EXAMPLE 3

### Tumor Cell Proliferation is Closely Correlated with Hexokinase Levels of Tumors

To examine the proliferative capacity of the tumor cells, each clonal isolate was seeded at 25×10³ cells per well in 24-well tissue culture plates in quadruplicate. The culture medium was replaced with DMEM (which lacks thymidine) for 24 hrs and then pulsed with 1.0 ml of the same medium containing 1 μCi/ml ³H-thymidine per well for 4 to 12 hrs. The cells were then washed in ice-cold PBS followed by ice-cold 10% TCA to remove unincorporated ³H-thymidine and acid-soluble material. Newly synthesized DNA in the acid-insoluble precipitate was dissolved in 0.3 M NaOH, 1% SDS. 50 μl aliquots of each sample was assayed for incorporated ³H-thymidine by scintillation counting. Total protein was assayed by the Bradford method using 500-fold dilutions of each sample.

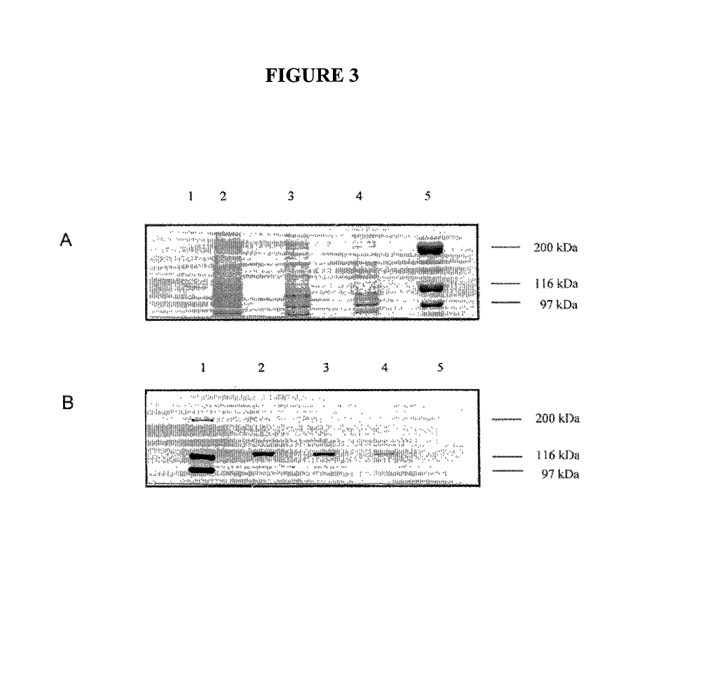

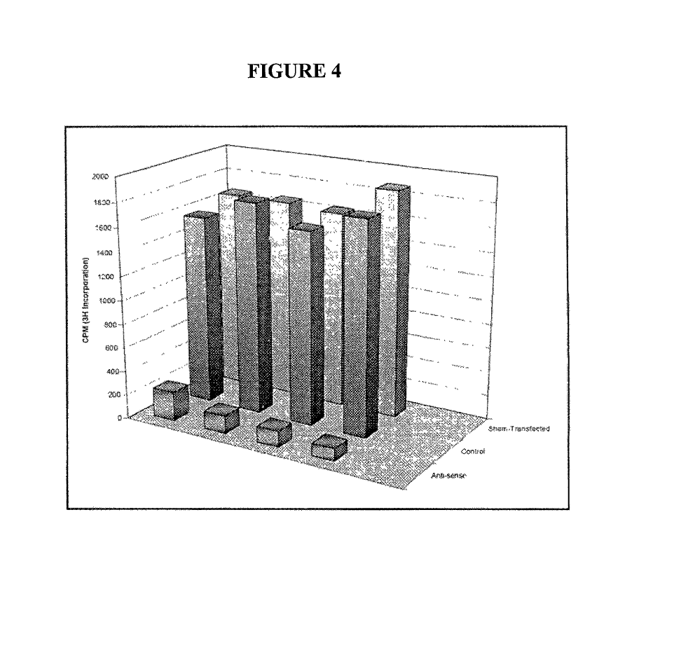

Several Type II hexokinase anti-sense clones were isolated with 82 to 93% reduction of hexokinase activity. Of these, the clone that showed the highest reduction in enzyme activity (93%) was analyzed by Western blotting (FIG. 3) to test for the hexokinase expression profile between the control and anti-sense cells. The ³H-thymidine incorporation assays, carried out in a pulse sequence of up to 12 hrs, also showed a marked decrease in their proliferation rate (88%, or approximately a 10-fold reduction) (FIG. 4).

### EXAMPLE 4

### Targeting Hexokinase is an Effective Therapeutic Strategy in Control of Tumor Cell Proliferation

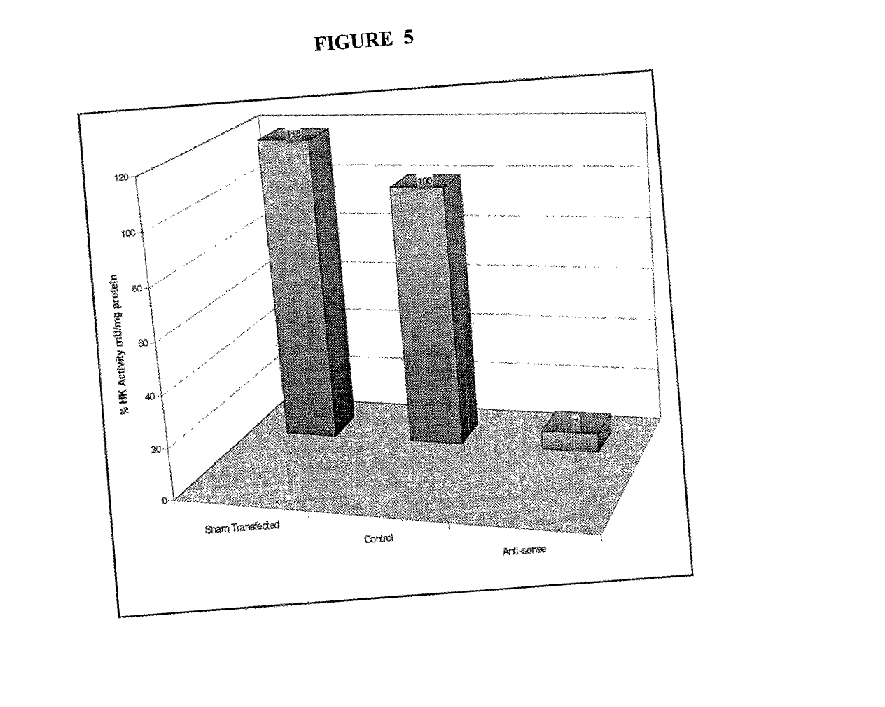

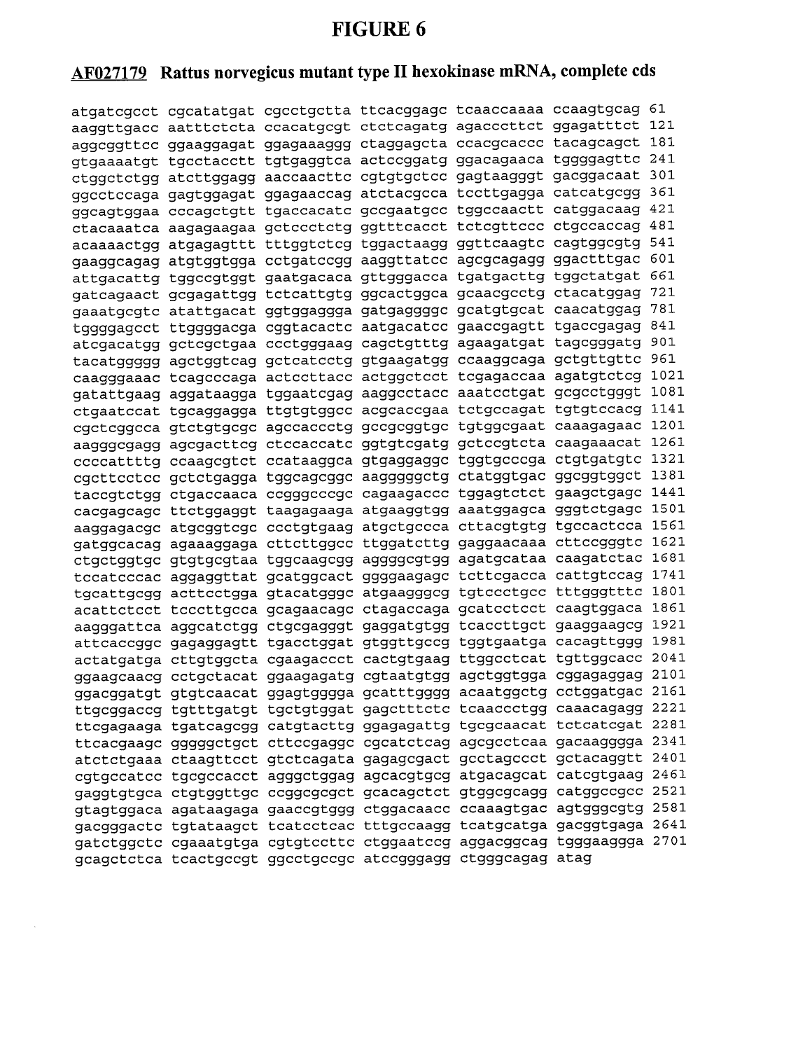

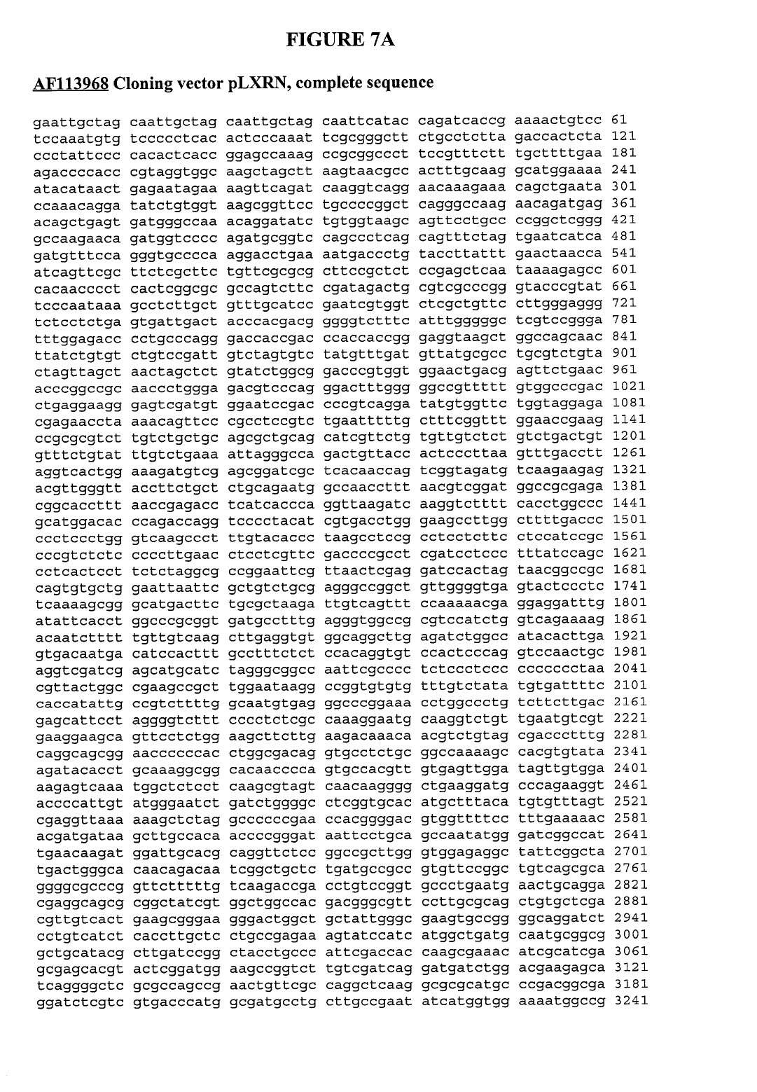

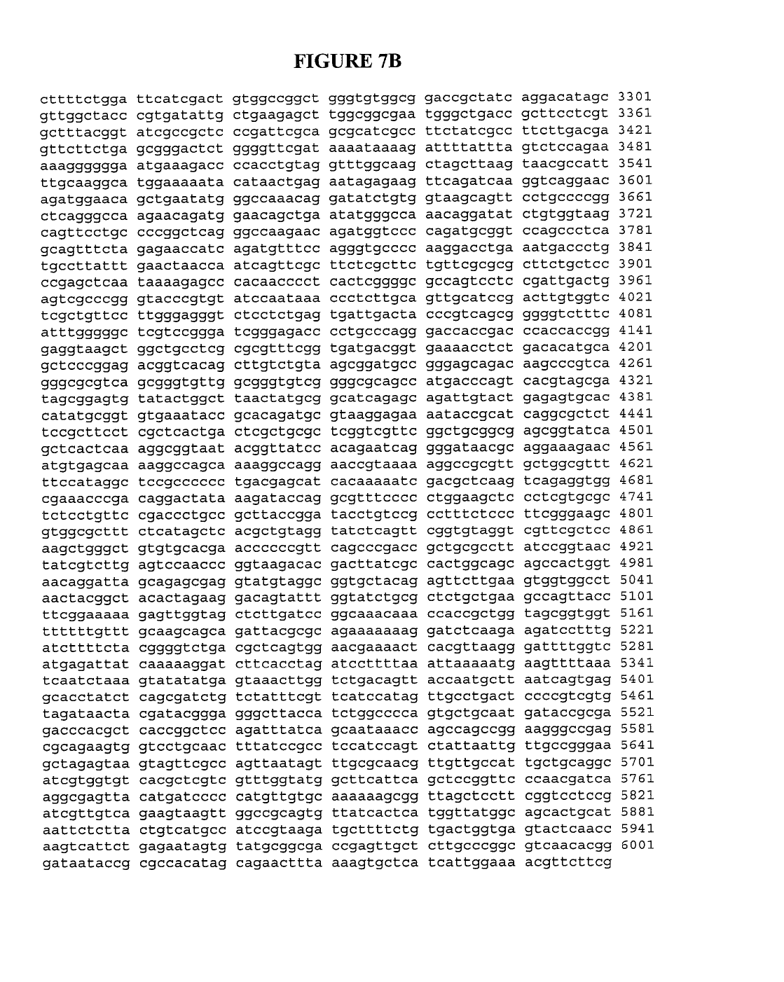

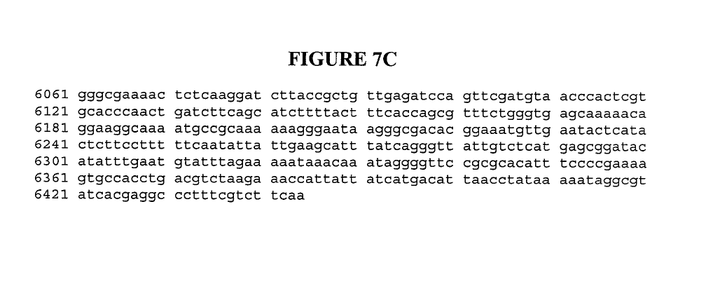

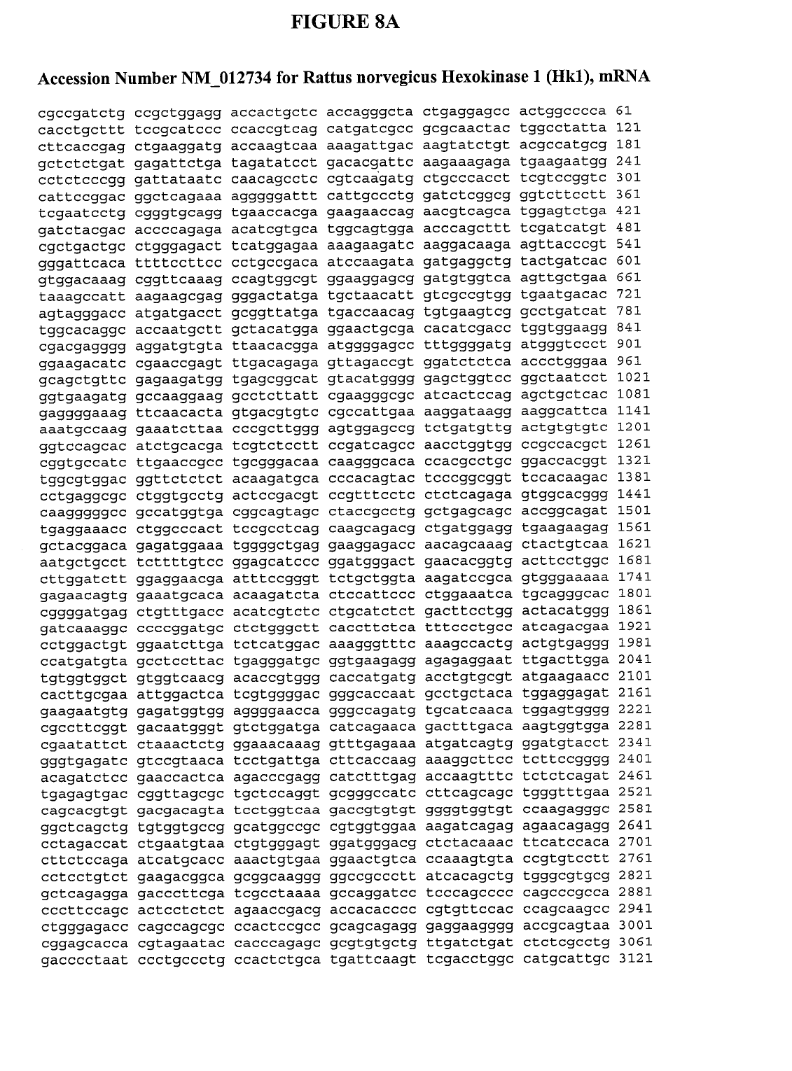

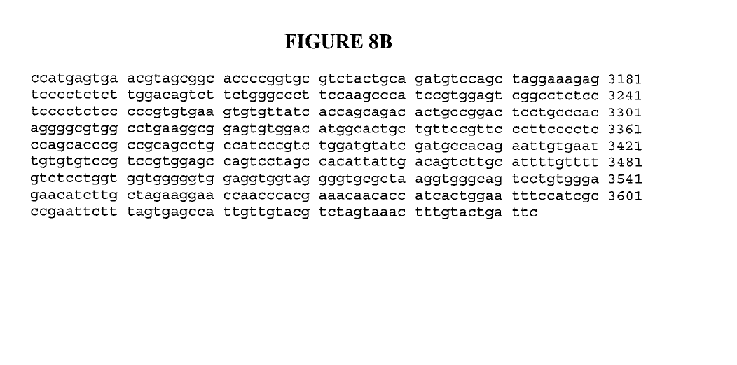

Since the type II hexokinase cassette used to generate the antisense template for the present study has close similarity to type I isoform of hexokinase also, it is highly likely that the current strategy was effective in targeting both isoforms in the tumor to reduce the phosphorylation of glucose. The elimination of these low Kₘ hexokinase pools (Type I and Type II hexokinase) from the highly glycolytic tumors has now shown a remarkable negative effect on their ability to proliferate (FIG. 5). This indicates an important requirement for hexokinase for the growth and survival of these cells.

Furthermore, during the analyses of clones, it has not been possible to locate a clonal isolate where the hexokinase activity was completely eliminated. Given the data obtained during the ³H-thymidine incorporation assays, it is quite possible that such “knock-outs”, even if generated immediately after the retroviral transfection procedure, were eliminated during the clonal selection if such “knock-outs” are “metabolically lethal” for these tumors.

The results of this study demonstrate the crucial nature of the hexokinase Type II gene for tumor survival and proliferation.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of inhibiting proliferation of tumor cells characterized by having a highly glycolytic phenotype comprising contacting the cells with a proliferation inhibitory effective amount of an antisense polynucleotide or oligonucleotide that hybridizes with a mRNA encoding a hexokinase under conditions that allow hybridization of the antisense polynucleotide with the mRNA, thereby inhibiting the proliferation of tumor cells.

2. The method of claim 1, wherein the mRNA encoding hexokinase has a nucleotide sequence complementary to a sequence as set forth in SEQ ID NO: 1.

3. The method of claim 1, wherein the hexokinase is Type II hexokinase.

4. The method of claim 1, wherein the hexokinase is Type I hexokinase.

5. The method of claim 1, wherein the mRNA encoding hexokinase has a nucleotide sequence complementary to a sequence as set forth in SEQ ID NO:2.

6. The method of claim 1, wherein the antisense polynucleotide or oligonucleotide comprises at least one modified intemucleoside linkage.

7. The method of claim 6, wherein the modified intemucleoside linkage is a phosphorothioate linkage.

8. The method of claim 1, wherein the antisense oligonucleotide comprises at least one modified sugar moiety.

9. The method of claim 8, wherein the modified sugar moiety is a 2′-O-methoxyethyl sugar moiety.

10. The method of claim 1, wherein the antisense oligonucleotide comprises at least one modified nucleobase.

11. The method of claim 10, wherein the modified nucleobase is a 5-methylcytosine.

12. The method of claim 1, wherein the antisense oligonucleotide is a chimeric oligonucleotide.

13. The method of claim 1, wherein the tumor cells are located in a tissue selected from the group consisting of brain, colon, urogenital, lung, renal, prostate, pancreas, liver, esophagus, stomach, hematopoietic, breast, thymus, testis, ovarian, and uterine tissue.

14. The method of claim 1, wherein prior to treating tumor cells with the antisense polynucleotide or oligonucleotide, the cells are diagnosed as highly glycolytic by obtaining a specimen selected from the group consisting of serum, urine, saliva, blood, cerebrospinal fluid, pleural fluid, ascites fluid, sputum, stool, bone marrow and biopsy sample.

15. The method of claim 1, wherein said cellular proliferative disorder is selected from the group consisting of low grade astrocytoma, anaplastic astrocytoma, glioblastoma, medulloblastoma, gastric cancer, hepatoma, colorectal cancer, colorectal adenoma, acute myelogenous leukemia, lung cancer, renal cancer, leukemia, breast cancer, prostate cancer, endometrial cancer, bone cancer, squamous cell cancer and neuroblastoma.

16. A method of modulating the expression of a hexokinase in a tumor characterized as having a highly glycolytic phenotype comprising contacting tumor cells or tissue with an antisense polynucleotide or oligonucleotide that hybridizes with the hexokinase encoding mRNA such that hexokinase enzyme translation is inhibited.

17. A pharmaceutical preparation useful for inhibiting proliferation of tumor cells comprising an antisense polynucleotide that hybridizes with a mRNA encoding a hexokinase in a pharmaceutically effective carrier.

18. The pharmaceutical preparation of claim 17, wherein the antisense polynucleotide has a sequence complementary to the sequence set forth in SEQ ID NO: 1.

19. A recombinant nucleic acid vector comprising the following elements operably linked in a 5′ to 3′ direction:
(a) a 5′ long terminal repeat (LTR) sequence;
(b) a multiple cloning site (MCS);
(c) an internal ribosome entry sequence (IRES);
(d) a selectable marker sequence; and
(e) a 3′ long terminal repeat (LTR) sequence.

20. The vector of claim 19, wherein the selectable marker sequence is a neomycin resistance sequence.

21. The vector of claim 19, wherein the vector has the sequence set forth in SEQ ID NO:3.

22. The vector of claim 19, further comprising a polynucleotide having the nucleotide sequence complementary to the sequence set forth in SEQ ID NO:1, said polynucleotide located 5′ of the IRES sequence.

23. A recombinant nucleic acid molecule comprising a polynucleotide complementary to a nucleotide sequence encoding Type II hexokinase.

24. The recombinant nucleic acid molecule of claim 23, wherein the polynucleotide has a nucleotide sequence complementary to the sequence set forth in SEQ ID NO:1.

25. A recombinant nucleic acid molecule comprising an polynucleotide complementary to a nucleotide sequence encoding Type I hexokinase.

26. The recombinant nucleic acid molecule of claim 25, wherein the polynucleotide has a sequence complementary to the nucleotide sequence set forth in SEQ ID NO:2.
